(19) United States
(12) Patent Application Publication
(10) Pub. No.: US 2011/0151028 A1
(43) Pub. Date: Jun. 23, 2011

(54) Topical vaginal preparation

(76) Inventors: Filiberto Zadini, Northridge, CA (US); Giorgio Zadini, (US)

(21) Appl. No.: 12/928,828

(22) Filed: Dec. 20, 2010

Related U.S. Application Data
(60) Provisional application No. 61/284,653, filed on Dec. 19, 2009.

(51) Int. Cl. A61K 36/00; A61K 38/11; A61K 31/19; A61K 31/215; A61K 31/221; A61P 15/02

(52) U.S. Cl. 424/725; 514/11.6; 514/573; 514/530; 514/551

(57) ABSTRACT

A topical pharmacological preparation in the form of cream, suppositories or drops delivered topically to the vagina in order to contract the smooth muscle of the vaginal wall. The preparation contains a smooth muscle contracting agent such as oxytocin capable of constrict the vaginal tube reducing its overall diameter for a more pleasurable sexual experience for the female subject and his partner alike. The smooth muscle contracting agent can be associated with a systemic absorption retardant such as a mixture of Benzyl Alcohol, Acetone and Isopropanol. Such a formulation achieves high local tissue concentration of the smooth muscle contraction substance such as oxytocin in vicinity to the site of application, via minimization of systemic absorption of the smooth muscle contraction inducing substance and at the same time via promotion of its submucosal absorption across the mucosal barrier. As a result of the use of such formulation an increased local smooth muscle contraction activity is expected to occur in the muscularis mucosae.

### RELATED MATTER

This application claims priority to U.S. Provisional Patent Application No. 61/284,653, filed on Dec. 19, 2009 and entitled “Topical Vaginal Preparation”, the relevant content of which is hereby incorporated by reference.

### FIELD OF INVENTION

This application relates to topical vaginal preparations aimed at achieving a time-controlled and strength-controlled contraction status of the smooth muscle of the vagina to improve vaginal tightness around the male copulation organ and, hence, enhance sexual satisfaction during copulation.

### BACKGROUND OF THE INVENTION

The vagina tends to lose its tonicity with age to a point that its laxity interferes negatively with intercourse. Besides age, numerous are the causes of loss of tone by the vagina: loss of tone may result from multiple childbirths, local traumas, chronic infections.

Loss of tone, i.e. increased laxity, of the vaginal canal results into reduced tightness of the vaginal canal around an erected penis which translates into diminished contact friction and hence less enjoyable intercourse.

### BRIEF SUMMARY OF THE INVENTION

Applicants disclose a topical preparation which when applied into the vagina and placed in contact with the vaginal walls, induces a short lived contraction of the smooth muscle of the vaginal walls causing overall tightening of the vaginal tube/canal. The topical preparation is in the form of cream or drops or dissolving vaginal tablets or suppositories. With proper dosage, contraction of the smooth muscle of the vagina can be controlled in term of time and strength of contraction.

In a improved embodiment the topical pharmacological compound is associated with a systemic absorption retardant to delay absorption of the vaginal muscle contraction inducer substance into the systemic circulation to minimize systemic absorption from the vaginal mucosa so as to maximize submucosal accumulation in the smooth muscle of the substance that induces vaginal muscle contraction in order to achieve maximization of local contraction effect and at the same time to minimize systemic absorption of the substance.

With the present Provisional Patent Application, Applicants disclose oxytocin as smooth muscle contracting agent for its record of safety and effectiveness in the obstetric-gynecological field. A list of other smooth muscle contractors is also disclosed including phytochemical compounds. Contraction may occur in a persistent constant fashion of limited duration or in waves depending upon the used muscle contractor, the means of administration, such as cream or vaginal suppositories or intravaginal drops, and the dose being administered.

A search in the patent office has not identified any patent disclosing a topical preparation containing smooth muscle contracting agent with the purpose of constricting the vaginal canal for intercourse. Applicants have identified few OTC—over the counter—topical preparations which act in a different way to tight the vaginal wall such as for instance increasing the water content of the vaginal walls.

These creams claim to tighten the vaginal walls to improve overall sexual satisfaction. None of the above compounds acts as a smooth muscle contractor/constricting agent.

### OBJECTS OF THE INVENTION

It is an object of the present invention to provide the female with a pharmacological compound capable of inducing sustained contraction of the vaginal wall with a purpose of increasing the contract-friction with an erected penis, resulting in a more pleasurable sexual experience for female and male alike.

### SPECIFICATIONS

The invention consists of a locally applied pharmacological compound which induces a sustained smooth muscle contraction of the walls of the vagina. In the preferred embodiment oxytocin is used in dose lower than the dose used systemically. Oxytocin is a time tested compound used systemically in the obstetric and gynecological field to cause uterine contractions. The vaginal preparation in form of tablets, suppositories, drops or cream is associated with a fatty base excipient.

In the cream preparation oxytocin is associated with fats and oils. The vehicle in the cream varies: it can contain, among other ingredients, benzoic acid, acetyl alcohol, isopropyl myristate, polysorbate 60, potassium hydroxide, propylene glycol, purified water and stearyl alcohol.

The recommended oxytocin dose is only a fraction of the dose used systemically.

Applicant propose a oxytocin cream concentration of 0.5% up to 2%. The sufficient dose to achieve contraction of the vagina will be in the order of milliunits, 1 to 4 milliunits to a maximum of 5 units.

Other pharmacological smooth muscle contractors are:

Carbachol, also known as carbamyicholine, which is a drug that binds and activates the acetylcholine receptor and is therefore classified as a cholinergic agonist. Other smooth muscle contracting agents are Angiotensin II, and d-Glutamic Acid. Among other smooth contracting agents are the Prostaglandins: PGE 2, Dinoprostone, at an intravaginal dose that Applicants propose to be ranging from 0.5 mg to 5 mg; PGE 1, Misoprostol, at an intravaginal dose that Applicants propose ranging from 25mcg to 400 mcg., PGF 2 alfa, at a dose yet to be established.

Other smooth muscle contracting agents are: Physiostigmine Salicylate or Sulphate Neostigmine Bromide or Methyilsulphate, Ambenomium Chloride, Piridostigmine Bromide, Edrophomium Clhoride, Demecarium Bromide, Echothiophate Iodide, Isofluorophate, Pradiloxime Chloride, Abidoxime Clhoride.

Other smooth muscle contracting agents that Applicants propose are derived from plants, and are

Tannic acid., dose to be established; Indigofera dendroides extracts, dose to be established; Ryanodine, which is a powerfull alkaloid smooth muscle contracting agentr found in the South American plant Ryania speciosa of the Flacourtiaceae. The effect of the ryanodine at the nanomolar-level is that ryanodine causes release of calcium from calcium stores in the sarcoplasmic reticulum leading to significant muscular contractions. Ryanodine potency is increased by the presence of caffeine. Another plant derived smooth muscle contracting agent is Emodin, which is a naturally occurring anthraquinone present in the roots and bark

of numerous plants of the genus Rhamnus. Extracts from the roots, bark, and/or dried leaves of buckthorn, senna, cascara,aloe, frangula, and rhubarb have been used as a smooth muscle contractors since ancient times and currently are widely used in laxative preparations

It is now understood that the contraction of smooth muscle cells involves two processes: (a) The concentration of intracellular Ca2+ increases; this Ca2+ increase results in phosphorylation of myosin and consequently an increased contractility. (b) The sensitivity of the myofilaments to Ca2+ increases. Pharmacological agents can stimulate smooth muscle cell contractions by mobilizing intracellular Ca2+ and/or enhancing Ca2+ sensitivity.

Applicants propose in another embodiment the addition of an absorption retardant to the muscle contracting agent.

The purpose for associating in the present application a smooth muscle contracting agent with an absorption retardant mixture is to deliver the smooth muscle contracting agent locally into the submucosal tissue minimizing systemic absorption from the tunica propria layer so as to maximize submucosal accumulation of smooth muscle contracting agent in order to achieve maximization of local muscle contracting effect in the muscularis mucosae layer.

Indeed, in situations in which an absorption retardant is not used, the tunica propria microvasculature may absorb the smooth muscle contracting agent and deliver it into the systemic circulation, removing it from the topical site where it is expected to exert its pharmacological effect, or even before it ever reaches the targeted muscularis mucosae layer of the vagina.

In the cited study conducted at USC, a mixture of Benzyl Alcohol, Acetone and Isopropanol has resulted not only with a promotion of intradermal delivery of the erythromycin, but also with a significant and persistent local accumulation of erythromycin in the dermal layer, while, on the contrary, the use of a standard hydrophilic transdermal carrier, such as propylene glycol, has resulted with a rapid systemic absorption of the transdermally delivered erythromycin and with a very negligible and short lived accumulation of the erythromycin in the dermal layer.

Applicants assert that such mixture of Benzyl Alcohol, Acetone and Isopropanol combined with the smooth muscle contracting agent will promote transport of the smooth muscle contracting inducer across the epidermal mucosal barrier while at the same time such a mixture will minimize systemic absorption with a resulting significant and persistent accumulation of the smooth muscle contraction inducer in the submucosal tissue including the muscularis mucosae layer tissue. Such a formulation has the potential for achieving high local tissue concentration in the vicinity of the site of application, and, consequently, it has the potential for maximizing the local pharmacological effects of the muscle contraction inducing substances in muscularis mucosae layer, in the proximity of the site of application enhancing its effect at the site of application. The respective concentration of Benzyl Alcohol, Acetone and Isopropanol varies, however, the recommended concentration is: benzyl alcohol (10%), acetone (40%), isopropanol (50%). Applicants propose the use of the mixture of Benzyl Alcohol, Acetone and Isopropanol in which at least two of the three components are present.

Other mucosa penetration enhancers which facilitate transport of the active principles thru the vaginal mucosa into the target tissue can be added to the topical vaginal preparation already containing the muscle contraction inducing ingredients and a submucosal absorption retardant such as the Benzyl Alcohol, Acetone and Isopropanol mixture.

The mucosa permeability enhancers that can be used include Percutaneous Chemical Enhancers

In this specific case it is logic to deduct that local accumulation of the muscle contracting inducing substances in the tunica propria and muscularis mucosae layer will enhance local effects of the active ingredients due to the fact that uptake of the therapeutic substance into the systemic circulation is delayed.

What we claim is:

1. A topical vaginal pharmacological preparation placeable in contact with the vaginal walls, said walls having a muscularis mucosa layer comprising:
a pharmacological compound capable of inducing a time controlled contraction of the smooth muscle of the vaginal walls causing overall tightening of the vaginal tube and
an excipient to vehiculate into the muscularis mucosa the pharmacological compound capable of inducing a short lived time controlled contraction of the smooth muscle of the vaginal walls.

2. The topical vaginal pharmacological preparation of claim 1 causing a strength controlled contraction of the smooth muscle of the vaginal walls causing overall tightening of the vaginal tube

3. The topical preparation of claim 2 wherein the excipient is a fatty based excipient.

4. The topical preparation of claim 2 wherein the pharmacological compound is in the form in the form of a cream.

5. The topical preparation of claim 1 wherein the pharmacological compound is in the form in the form of intravaginal drops.

6. The topical preparation of claim 1 wherein the pharmacological compound is in the form in the form of intravaginal dissolving tablets.

7. The topical preparation of claim 2 wherein the pharmacological compound is in the form in the form of intravaginal suppositories.

8. The topical preparation of claim 2 wherein the pharmacological compound is oxytocin.

9. The topical preparation of claim 8 wherein oxytocin is in doses ranging from 1 to 5 units.

10. The topical preparation of claim 2 wherein the pharmacological compound is a Prostaglandin.

11. The topical preparation of claim 9 wherein the prostaglandin is a PGE 2 prostaglandin, Dinoprostone at an intravaginal dose that ranging from 0.5 mg to 5 mg.

12. The topical preparation of claim 9 wherein the prostaglandin is Misoprostol, at an intravaginal dose ranging from 25 mcg to 400 mcg.,

13. A topical preparation comprising a pharmacological compound acting as a contractor of the smooth muscle of the vaginal walls causing a sustained time controlled decrease in diameter of the vaginal tube and a fatty based excipient.

14. The topical preparation of claim 2 comprising an absorbent retardant of the pharmacological compound capable of inducing contraction of the smooth muscle of the vaginal walls causing overall tightening of the vaginal tube, said absorbent retardant minimizing systemic absorption and maximizing local accumulation.

15. The absorbent retardant of claim 13 comprising a mixture of Benzyl Alcohol, Acetone and Isopropanol.

16. The topical preparation of claim 2 wherein the pharmacological compound is carbachol.

17. The topical preparation of claim 2 wherein the pharmacological compound is a plant derived compound
